(19) United States
(12) Patent Application Publication
(10) Pub. No.: US 2003/0113800 A1
(43) Pub. Date: Jun. 19, 2003

(54) Materials for screening of combinatorial libraries

(76) Inventors: Klaus Mosbach, Furulund (SE); Olof Ramstrom, Lund (SE); Lei Ye, Lund (SE)

Correspondence Address: Benton S. Duffett, Jr., BURNS, DOANE, SWECKER & MATHIS, L.L.P., P.O. Box 1404, Alexandria, VA, 22313-1404, US

(21) Appl. No.: 10/263,195

(22) Filed: Oct. 3, 2002

Related U.S. Application Data
(63) Continuation of application No. 09/607,925, filed on Jun. 30, 2000.
Application No. 09/607,925 is a continuation of application No. PCT/SE98/02413, filed on Dec. 22, 1998.

(30) Foreign Application Priority Data
Dec. 30, 1997 (SE) 9704919-1

(51) Int. Cl. C12Q 1/68; G01N 33/53; C07J 53/00; G01N 33/543

(52) U.S. Cl. 435/7.1; 436/518; 552/500

(57) ABSTRACT

This Invention refers to the use of molecularly imprinted polymers as a method in which the selectivities of imprinted materials can be gainfully employed as binding matrices in the screening of combinatorial libraries.

### BACKGROUND OF THE INVENTION

In recent years, molecular imprinting has become an increasingly attractive approach for mimicking natural binding events 1-5, It offers an effective means of preparing recognition materials with binding properties that resemble those of natural binding entities such as antibodies and receptors 6-9. By using the same types of molecular interactions present in bio-affinity processes (e.g., ionic interactions, hydrogen bonds, and hydrophobic interactions), tailor-made affinity materials can be designed for practically any chosen target substance.

The binding performances of molecularly-imprinted polymers (MIPs) give these materials great potential in combinatorial approaches as recognition matrices for the screening and rapid selection of ligands from a combinatorial library 10. The high selectivity that can be obtained, in conjunction with the robustness, are features that make MIPs especially suitable for this type of application 11 It occurred to us that these specific adsorbents could be useful in the screening of chemical combinatorial libraries (CCLs) 12, and biological combinatorial libraries 13

SHORT DESCRIPTION OF THE DRAWINGS

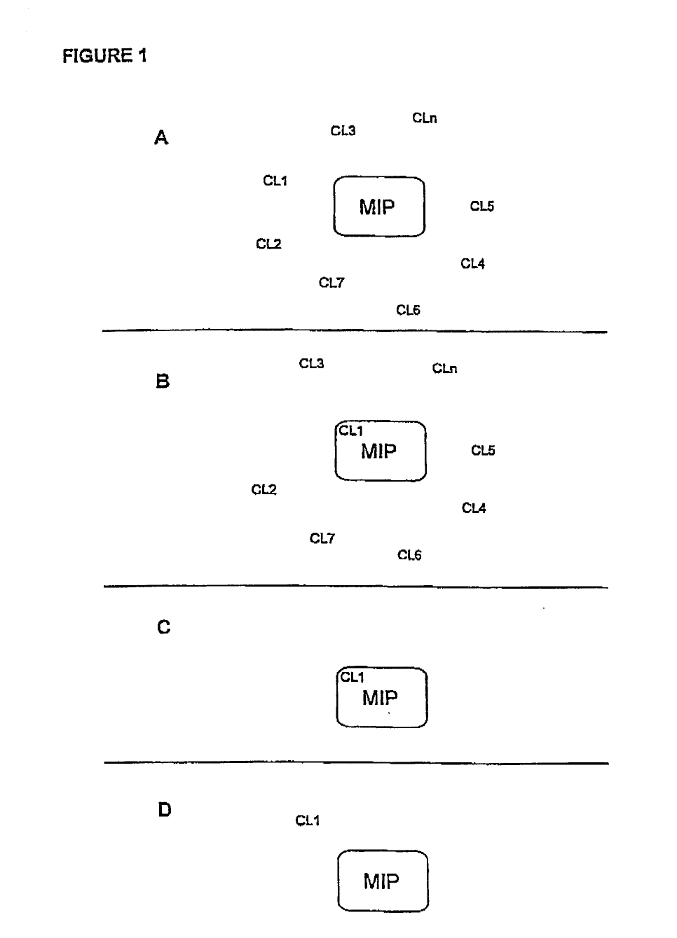

FIG. 1 describes the use of a molecularly imprinted :polymer (MIP) in selective binding and screening of a compound from a combinatorial library. A) The compounds of the combinatorial library (CL1, CL2 . . . CLn) are allowed to interact with the MIP. B) One selected compound of the library (CL1) binds more strongly to the MIP than any of the others. C) The compounds of the library that are unbound (CL2, CL3 . . . CLn) by the MIP can be washed away. D) The bound species (CLI) can be extracted.

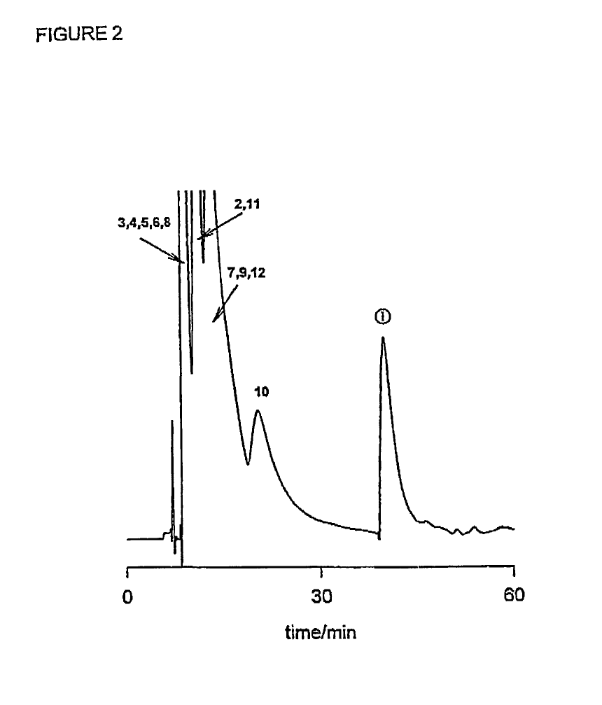

FIG. 2 shows the screening of a steroid library using a MIP according to example 1. MIP prepared against 11-α-hydroxyprogesterone (1). Gradient elution: 0-25 min, dichloromethane 0.1% acetic acid (v/v); 25-30 min, dichloromethane 0.1%-5% acetic acid (v/v); 30-40 min, dichloromethane 5% acetic acid (v/v), 40-45 min, dichloromethane 5%-0.1% acetic acid (v/v); 0.5 mL/min; Sample 20 μL, concentration; 0.8 mM of each component. Average of two consecutive analyses. The numbering of the species (1-12) are as follows: 11α-Hydroxyprogesterone (1), 11β-Hydroxyprogesterone (2), 17α-Hydroxyprogesterone (3), Progesterone (4), 4-Androsten-3,17-dione (5), 1,4-Androstadiene-3,17-dione (6), Corticosterone (7), Cortexone (8), 11-Deoxycortisol (9), Cortisone (10), Cortisone 21 -acetate (11), Cortisol 21-acetate (12)

### DETAILED DESCRIPTION OF THE INVENTION

In the following, we would like to describe an invention addressing the use of MIPs in selective binding and screening of compounds from a combinatorial library. The principle is outlined in FIG. 1, where CL1, CL2 . . . CLn, represent compounds of a combinatorial library composed of n different compounds, and MIP represents a molecularly imprinted polymer selective for compound CL1. In the first step (step A), the compounds of the combinatorial library is allowed to freely interact with the MIP. Under these conditions, one of the compounds of the library (CL1) binds more strongly to the HIP (as selected from the MIP-preparation) than any of the others (step B). In the subsequent step (step C), the remaining, not bound compounds of the library (CL2, CL3 . . . CLn) can be washed away from the system. Finally, (step D), the compound of the library that bound to the MIP (CL1) can be extracted. In this way, the MIP is used as a selective screening matrix for a selected compound from a combinatorial library.

Another, non-limiting use of this methodology is the use of MIPs for simultaneous binding of a group of molecules from a library of related structures. By using several compounds in the MIP preparation, several compounds can be selectively bound to the HIP. Similarly, a MIP prepared against one compound can be used to selectively bind a group of compounds tram a library.

### EXAMPLE 1

In this example, the technique was demonstrated using a chemical combinatorial library. The combinatorial steroid library used in the example is displayed in Table 1. The library was composed of twelve closely related androsten-3-one structures, differing only at positions 1, 11, and 17 (including sidechain). Two compounds from the library were chosen as target molecules, 11-α-hydroxyprogesterone (1), and corticosterone (7), and used in the preparation of MIPs (anti-1-MIP and anti-7-MIP, respectively). Control polymers were prepared, using the same imprinting protocol, in the absence of any template steroids.

The resulting MIPS were subjected to a work-up protocol and subsequently packed into HPLC columns. In order to verify the order of elution, and to estimate the specificity of the MIPs, individual administrations of the library species were performed. The results from the chromatographic evaluation of the binding specificities are displayed in Table 2. As can be seen from these figures, it is clear that both types of MIPs displayed high specificity with respect to their respective template species. Anti-l-MIP retained 11-α-hydroxyprogesterone longer than any other compound in the library, and anti-7-MIP showed a similar behavior with respect to corticosterone. As a comparison, these compounds were not substantially retained by the control polymers.

TABLE 1
Steroid structures

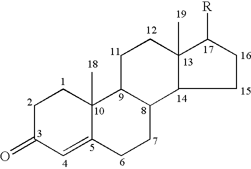

| | | Other |
|---|---|---|
| Compound | R | substituents |
| 11α-Hydroxyprogesterone (1) | COCH₃ | 11α-OH |
| 11β-Hydroxyprogesterone (2) | COCH₃ | 11β-OH |
| 17α-Hydroxyprogesterone (3) | COCH₃ | 17α-OH |
| Progesterone (4) | COCH₃ | |
| 4-Androsten-3,17-dione (5) | ═O | |
| 1,4-Androstadiene-3,17-dione (6) | ═O | Δ¹ |
| Corticosterone (7) | COCH₂OH | 11β-OH |
| Cortexone (8) | COCH₂OH | |
| 11-Deoxycortisol (9) | COCH₂OH | 17α-OH |
| Cortisone (10) | COCH₂OH | 11═O, 17α-OH |
| Cortisone 21-acetate (11) | COCH₂OAc | 11═O, 17α-OH |
| Cortisone 21-acetate (12) | COCH₂OAc | 11β-OH, 17α-OH |

TABLE 2
Binding specificities. Retention indices using
individual injections of 1 mM samples of the library
components. Isocratic elution: DCM 0.1% acetic acid
(anti-1-MIP), DCM 0.5% acetic acid (anti-7-MIP).

| Compound | anti-1-MIP | anti-7-MIP |
|---|---|---|
| 11α-Hydroxyprogesterone (1) | 100 | 11 |
| 11β-Hydroxyprogesterone (2) | 13 | 22 |
| 17α-Hydroxyprogesterone (3) | 10 | 8 |
| Progesterone (4) | IS^{a} | IS^{a} |
| 4-Androsten-3,17-dione (5) | −3 | 0 |
| 1,4-Androstadiene-3,17-dione | 4 | 5 |
| Corticosterone (7) | 11 | 100 |
| Cortexone (8) | 7 | 41 |
| 11-Deoxycortisol (9) | 8 | 16 |
| Cortisone (10) | 10 | 12 |
| Cortisone 21-acetate (11) | 7 | 6 |
| Cortisol 21-acetate (12) | 8 | 10 |

^{a}Internal Standard

The results clearly demonstrate the efficiency of the imprinting process. When using 11-α-hydroxyprogesterone (1) as a print species, this compound could be easily distinguished from the 11-α-isomer and the 17-α-isomer. Also, the anti-1-MIP could separate the print species from corticosterone (7) and cortisone (10), which were clearly more tightly retained by the control polymers. The results are indicative of a high importance for the presence and position of a hydroxyl group in the 11-position inasmuch as 11-β-hydroxyprogesterone (2) showed a substantially lower retention index than the print species did. On the other hand, the anti-7-MIP could efficiently separate corticosterone (7) from cortisone (10) and 11-deoxycortisol (9), both of which were more tightly retained by the control polymers. In this case, the absence of the hydroxyl group in the 21-position (sidechain) resulted in a major binding difference, whereas the absence of the 11-hydroxy group resulted in considerably higher crossbinding to the sites. Nevertheless, the recorded crossreactivities were very low in all cases.

The screening capability of the MIPs was estimated upon administration of the whole library onto the MIPs. The results from screening the library using the anti-1-MIP are displayed in FIG. 2. The results clearly indicate that the polymers were capable of selectively retaining the template species when offered the MIPs. Thus, the anti-1-MIP was capable of distinguishing 11-α-hydroxyprogesterone (1) from the library, and the anti-7-MIP could selectively bind corticosterone (7). In comparison, the non-imprinted control polymers showed no significant selectivity, and both print species were eluted well before the most tightly retained compound (cortisone, 10). Thus, in consequence of the molecular imprinting event, specific sites were introduced into the polymers that were capable of selectively fishing out the desired compounds from the library. In spite Of a close resemblance between the substances, it was possible to achieve enough specificity to distinguish small structural differences. These results indicate that MIPs can be successfully used as synthetic receptors in the screening of combinatorial libraries.

Experimental

The steroid library was purchased from Sigma (St. Louis, Mo., USA) and used as delivered. Methacrylic acid (MAA, dried over CaCl₂, distilled), ethylene glycol dimethacrylate (EDMA, dried over CaHl_{2, CaCl}₂, distilled), and azobis-isobutyronitrile (AIBN, used as delivered) were from Merck (Darmstadt, Germany). Dichloromethane (DCM, anhydrous) used in the imprinting protocol was from Lab-Scan (Stillorgan, Ireland). All other solvents were of HPLC-grade and used as delivered.

Polymers were prepared using two different print molecules (11-α-hydroxyprogesterone, 1, and corticosterone, 7), and MAA as a functional monomer. In a typical example, the print molecule (2.0 mmol), the functional monomer (12 mmol), the crosslinker (EDMA, 60 mmol), and the initiating agent (AIBN, 0.7 mmol) were mixed and dissolved in the porogen (dry DCM, 18 mL). The solutions were subsequently purged with nitrogen for 10 minutes and left to polymerize in a Rayonet photochemical reactor (Southern New England Ultraviolet Co., Bradford, CT, USA) at 350 nm at 4° C. for 16 hours. Each polymer was ground with a mechanical mortar (Retsch, Haan, Germany) and sieved through a 0.025-mm sieve (Retsch). Following repeated sedimentation in acetone, polymer particles ranging from approximately 0.01 to 0025 mm were collected. Control polymers were prepared, using the same protocol, in the absence of any print molecule.

Each polymer was slurry-packed into a stainless steel HPLC column (250×46 mm), and washed on-line with methanol/acetic acid (7:3) until a stable baseline was obtained. All analyses were performed using a Pharmacia-LKB type 2249 solvent delivery system equipped with a variable wavelength monitor model 2141 (Pharmacia-LKB Biotechnology, Uppsala, Sweden). Chromatographic analyses were performed either isocratically with DCM 0.1%/0.5% acetic acid (v/v), or using gradient elution with DCM 0.1% to 5% acetic acid at 0.5 mL/min at ambient temperature. Analytes were monitored by UV absorption at 240 nm using progesterone as an internal standard. Capacity factors,(k′), and retention indices (R.I.) were calculated using standard chromatographic theory 14,15. The retention index is a measure of the relative retention of the analytes with respect to both imprinted and control polymers, resulting in a value of 100% for the template species. R.I.={k′_{analyte}(MIP)/k′_{analyte}(control)}/{k′ₜₑₘₚₗₐₜₑ(MIP)/k′^{templa te}(control)}.

### REFERENCES

1 Mosbach, K., and Ramström, O., Bio/Technology, 1996, 14, 163.

2 Wulff, G., Angew. Chem. Int. Ed. Engl., 1995, 34, 1812.

3 Vidyasankar, S., and Arnold, F. H., Curr. Opin. Biotechnol., 1995, 6, 218.

4 Shea, K. J., Trends Polym. Sci. (Cambridge, U. K.), 1994, 2, 166.

5 Whitcombe, M. J., Alexander, C., and Vultson, E. N, Trends Food Sci. Technol., 1997, 6, 140.

6 Ramström, O., Ye, L., and Mosbach, K., Chem Biol, 1996, 3, 471.

7 Andersson, L. I., Muller, a., Vlatakis, G., and Mosbach, K., Proc. Natl. Acad. Sci. U.S.A., 1995, 92, 4788.

8 Andersson, L. I., Anal. Chem., 1996, 68, 111.

9 Kempe, M., Anal. Chen., 1996, 68, 1948.

10 Fenniri, H., Curr. Med. Chem., 1996, 3, 343.

11 Ramström, O., and Ansell, R. J., Chirality, 1998, in press.

12 Blondelle, S. E., Pérez-Payá, E., Dooley, C. T., Pinilla, C., and Houghten, R. A., Trends Anal. Chem., 1995, 14, 83.

13 Smith, G. P., and Petrenko, V. A., Chem. Rev., 1997, 97, 349.

14 Glad, M., Norrlöw, O., Sellergren, B., Siegbahn, N., and Mosbach, K., J. Chromatogr., 1985, 347, 11.

15 Allen ark, S. Chromatographic Enantioseparation: Methods and Applications; Ellis Horwood Limited: Chichester, 1988.

1. Use of a molecularly imprinted polymer for specific removal of a compound from a mixture of related structures representing a combinatorial library.

2. Use of a molecularly imprinted polymer for screening of a combinatorial library.

3. Use of a molecularly imprinted polymer for specific removal of a compound from a mixture of related structures representing a combinatorial chemical library.

4. Use of a molecularly imprinted polymer for specific removal of a compound from a mixture of related structures representing a combinatorial biological library.

5. Use according to claims 1-3 for Screening of a chemical library made up of steroids.

6. Use according to claims 1-3 for screening of a peptide library.
